(19) United States
(12) Patent Application Publication
(10) Pub. No.: US 2021/0220238 A1
(43) Pub. Date: Jul. 22, 2021

(54) SINGLE-SERVE ORAL RINSE SYSTEMS

(71) Applicant: Brian Morrow, Cambria, CA (US)

(72) Inventor: Brian Morrow, Cambria, CA (US)

(21) Appl. No.: 17/220,776

(22) Filed: Apr. 1, 2021

Related U.S. Application Data
(62) Division of application No. 16/656,327, filed on Oct. 17, 2019.
Application No. 16/656,327 is a continuation-in-part of application No. 15/064,795, filed on Mar. 9, 2016, now abandoned.
(60) Provisional application No. 62/218,691, filed on Sep. 15, 2015.

(51) Int. Cl. A61K 8/20; A61K 8/43; A61K 8/33; A61Q 11/00; A61K 8/49; A61K 8/21; A61K 8/34; A61K 8/19; A61K 8/41

(52) U.S. Cl. CPC A61K 8/20; A61K 8/43; A61K 8/33; A61Q 11/00; A61K 8/4926; A61K 2800/87; A61K 8/347; A61K 8/19; A61K 8/416; A61K 8/41; A61K 8/21

(57) ABSTRACT

A single-dose package that provides an appropriate amount of flavoring and an orally active germicide such as household bleach or sodium hypochlorite useful for a single batch of water-flosser rinse. The mixture eliminates the taste that occurs. It uses a mixture of an orally active germicide mixed with a flavoring such as mint, peppermint, wintergreen, anise, clove, eucalyptus, lemon, lime, honey, honey lemon, red fruit, grapefruit, orange, grape, cherry, cherry cola, berry, or cinnamon. Some mixtures include fluoride. Single-serve packaging helps water-flosser users receive appropriate amounts of bleach. The mixture can contain other excipients, solid or liquid carriers, and can come in powder, granule, gel, liquid, or tablet form. Other useful orally active germicides include glutaraldehyde, chlorhexidine, diamines, alexidine, phenols, formaldehyde, glutaraldehyde, acridines, silver compounds, halogens, silver ions, hexachlorophene, benzalkonium chloride, and their mixtures.

### CROSS-REFERENCE TO RELATED APPLICATION

The present application is a divisional of Ser. No. 16/656,327, filed on Oct. 17, 2019, pending, which is a continuation-in-part of U.S. utility application Ser. No. 15/064,795, filed on Mar. 9, 2016, abandoned, which claims priority to prior provisional application Ser. No. 62/218,691, filed Sep. 15, 2015, expired, all of which are incorporated herein by reference.

### BACKGROUND OF THE INVENTION

The following includes information that may be useful in understanding the present inventions. It is not an admission that any of the information provided herein is prior art, or material, to the presently described or claimed inventions or that any publication or document specifically or implicitly referenced is prior art.

### FIELD OF THE INVENTION

The present invention generally relates to dental hygiene products and, more specifically, relates to a single-serve oral rinse system.

### DESCRIPTION OF RELATED ART

People use oral irrigation devices as an alternative to flossing. These devices eliminate food particles from in-between teeth more effectively than traditional flossing. Water flossing can be more effective than typical dental floss for reducing gingivitis, reducing gingival bleeding, and removing plaque or germs below the gum line. It can also be difficult, inconvenient, and unsafe for an individual to measure and pour their bleach and/or flavoring. A suitable solution is desired.

Several attempts have been made to solve the above-mentioned problems such as those found in U.S. Pub. Nos. 2006/0073190 to Thomas Carroll; 2009/0260997 to Frank Vovelli; 2004/0091431 to Carol Alexander; 2004/0223921 to Allen Rau, U.S. Pat. No. 5,445,825 to Phoebe Copelan; and U.S. Pat. No. 6,887,493 to Adi Shefer. This art is representative of dental hygiene products. However, none of the above inventions and patents, taken either singly or in combination, is seen to describe the invention as claimed.

Preferably, a single-serve oral rinse system should eliminate the unpleasant taste that occurs when using bleach without flavoring while flossing teeth using an oral irrigation device and yet would operate reliably and be manufactured at a modest expense. Thus, a need exists for a reliable single-serve oral rinse system to avoid the above-mentioned problems.

### BRIEF SUMMARY OF THE INVENTION

In view of the foregoing disadvantages inherent in the known dental hygiene product art, the present invention provides a novel single-serve oral rinse system. The general purpose of the present invention, which will be described subsequently in greater detail, is to provide a single-serve oral rinse system.

Methods of using single-dose package that provides an appropriate amount of flavoring and an orally active germicide such as household bleach or sodium hypochlorite useful for a single batch of water-flosser rinse. The mixture eliminates the taste that occurs. It uses a mixture of an orally active germicide mixed with a flavoring such as mint, peppermint, wintergreen, anise, clove, eucalyptus, lemon, lime, honey, honey lemon, red fruit, grapefruit, orange, grape, cherry, cherry cola, berry, or cinnamon. Some mixtures include fluoride. Single-serve packaging helps water-flosser users receive appropriate amounts of bleach. The mixture can contain other excipients, solid or liquid carriers, and can come in powder, granule, gel, liquid, or tablet form. Other useful orally active germicides include glutaraldehyde, chlorhexidine, diamines, alexidine, phenols, formaldehyde, glutaraldehyde, acridines, silver compounds, halogens, silver ions, hexachlorophene, benzalkonium chloride, and their mixtures.

Disclosed is a breath freshening packet design for single-serve oral rinse application into a Waterpik or similar oral cleaning device. The packet of the current invention may include proper dosing of bleach with mint, peppermint, wintergreen, and cinnamon flavor in different dosages. A user may tear open the package and add contents into the Waterpik reservoir along with water. Breath freshening packet mixture may comprise a possible combination of bleach, mint, peppermint, wintergreen, and cinnamon, along with a single-use tear open packet in combination. The invention may be in the form of a single-serve tear open package, twist-off tab, or a single-serve dissolving tablet.

A single-serve oral rinse system is disclosed herein, in a preferred embodiment, comprising a flavored bleach composition having bleach, fluoride, and at least one flavor. Single-serve oral rinse system comprises the flavored bleach composition as an admixture. The admixture comprises at least one fluid. The admixture comprises at least one granule. The flavored bleach composition comprises in functional combination the bleach, the fluoride, and the at least one flavor. The at least one flavor may comprise mint; peppermint; wintergreen; and/or cinnamon or the like. Many flavors may be used, and the ones provided herein are merely exemplary and do not limit the scope of the present invention. The flavored bleach composition may further comprise a fluoride-additive.

The single-serve oral rinse system comprises a disposable packet for one-time use for containing the flavored bleach composition. The single-serve oral rinse system comprises tablet form for individual use in alternate embodiments. The flavored bleach composition is suitably mixed to provide a means for cleaning an oral surface using an oral irrigation device. Other devices may be used. The flavored bleach composition is non-toxic in-use. The oral surface (teeth, gums, tongue, etc.) is able to be cleansed of food particles. The oral surface is able to be cleansed of plaque. The at least one oral surface is able to be cleansed of germs. The single-serve oral rinse system reduces gingival bleeding and gingivitis.

A kit is described herein including a set of packets of said flavored bleach composition of at least one flavor and a set of user instructions.

A method of using a single-serve oral rinse system is also disclosed herein comprising the steps of opening a packet of a flavored bleach composition, pouring flavored bleach composition into an oral irrigation device, cleaning at least one oral surface, and disposing of packaging. Tablets may alternately be used.

The present invention holds significant improvements and serves as a single-serve oral rinse system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood by referencing the following drawings and detailed description.

### BRIEF DESCRIPTION OF THE DRAWINGS

The figures which accompany the written portion of this specification illustrate embodiments and methods of use for the present invention, single-serve oral rinse system, constructed and operative according to the teachings of the present invention.

FIG. 1 shows a perspective view illustrating a single-serve oral rinse system during an ‘in-use’ condition showing a packet of the single-serve oral rinse system being deposited into a water flosser according to an embodiment of the present invention.

FIG. 2 is a perspective view illustrating the (unopened) packet (labeled Breath Fresh) of the single-serve oral rinse system according to an embodiment of the present invention of FIG. 1.

FIG. 3 is a perspective view illustrating the unopened packet according to an embodiment of the present invention of FIGS. 1-2.

FIG. 4 is a perspective view illustrating a kit for the single-serve oral rinse system according to an embodiment of the present invention of FIGS. 1-3.

FIG. 5 is a flowchart illustrating a method of use for the single-serve oral rinse system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to dental hygiene products and, more particularly, to a single-serve oral rinse system as used to improve the efficiency of flossing the teeth of a user using an oral irrigation device.

Generally speaking, the present invention provides the exact amount of flavoring and bleach needed for a water flosser. As developed, it eliminates the unpleasant taste that occurs when using bleach without flavoring and is offered in a single-use, disposable packaging for ease of use. In some embodiments, this material utilizes a mixture with a possible combination of bleach, mint, peppermint, wintergreen, cinnamon, or a fluoride additive. It is designed and mixed to ensure that water-flosser users receive the appropriate amount of bleach.

This innovative product may comprise a mixture of bleach and fluoride with mint, peppermint, wintergreen, and/or cinnamon flavors. The product may come in small plastic or similar material packaging that may measure approximately 1⅞″×3¾″, or about the same size as a packet of soy sauce. It may be available in boxes containing enough packets for a week (7), two weeks (14), or more. There may be roughly a half teaspoon of the product in each packet, designed to be mixed into 10 oz of water. This bleach packet may also be available in an individual use tablet form (Alka-Seltzer style).

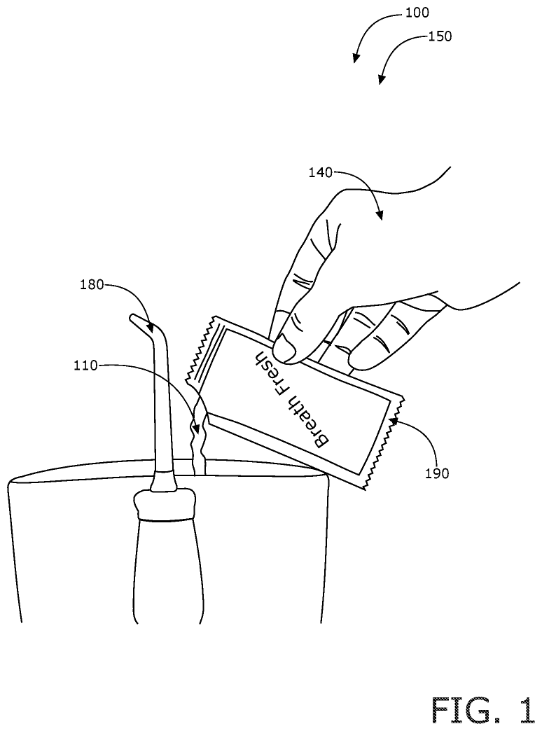

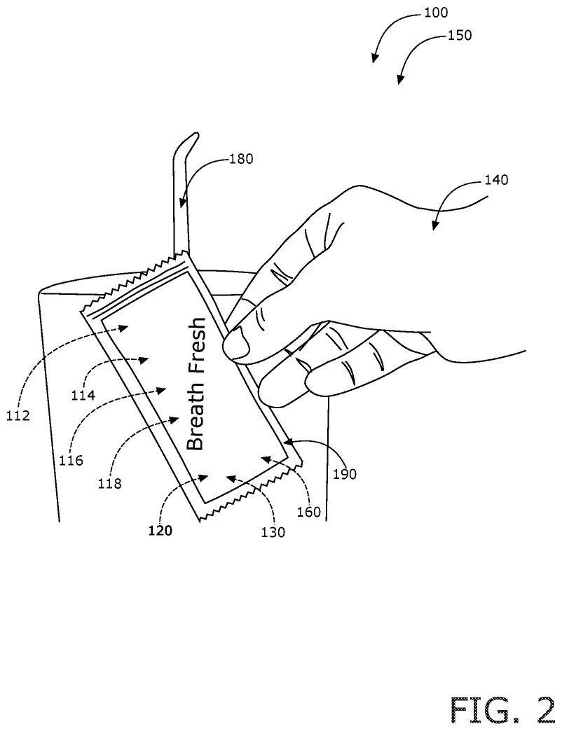

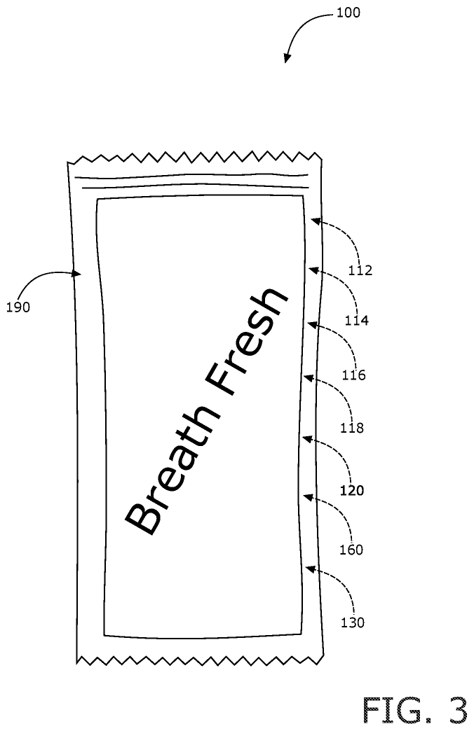

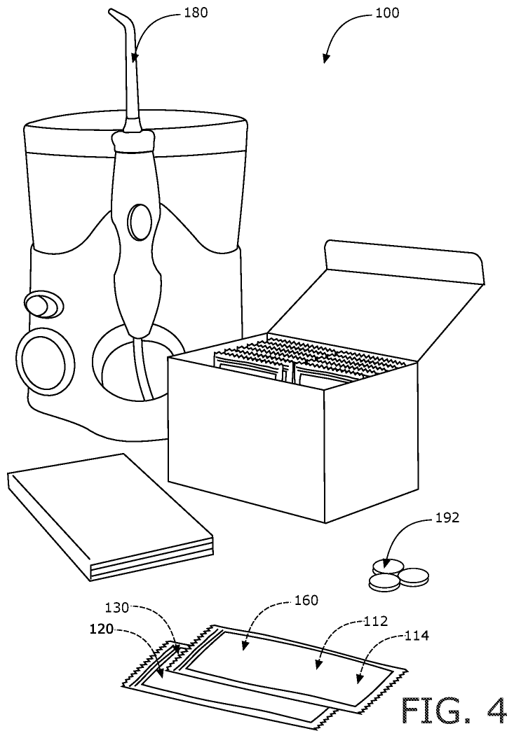

FIGS. 1-4 show single-serve oral rinse system 100 comprising flavored bleach composition 110 having bleach 120, fluoride 130, and at least one flavor 160. Single-serve oral rinse system 100 comprises flavored bleach composition 110 as admixture 112. Flavored bleach composition 110 comprises in functional combination bleach 120, fluoride 130, and at least one flavor 160. Flavored bleach composition 110 is suitably mixed to provide a means for cleaning at least one oral surface using oral irrigation device 180. Admixture 112 comprises at least one fluid 114. Admixture alternately may comprise at least one granule 116. At least one flavor 160 comprises mint, peppermint, wintergreen, and cinnamon or the like. Flavored bleach composition 110 further comprises fluoride-additive 118. In some embodiments, bleach is sodium hypochlorite or a similar oxygenated halide compound.

Single-serve oral rinse system 100 comprises disposable packet 190 for one-time use for containing flavored bleach composition 110. Single-serve oral rinse system 100 alternately comprises tablet form 192 for individual use. In some embodiments, flavored bleach composition 110 is non-toxic in-use. At least one oral surface is able to be cleansed of food particles. At least one oral surface is able to be cleansed of plaque. At least one oral surface is able to be cleansed of germs. Single-serve oral rinse system 100 reduces gingival bleeding. Single-serve oral rinse system 100 reduces gingivitis.

Single-serve oral rinse system 100 may be sold as a kit comprising the following parts: at least one set of packets of flavored bleach composition 110 of at least one flavor 160; and at least one set of user instructions. The kit has instructions such that functional relationships are detailed in relation to the structure of the invention (such that the invention can be used, maintained, or the like in a preferred manner). Single-serve oral rinse system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different flavoring combinations, parts may be sold separately, etc., may be sufficient.

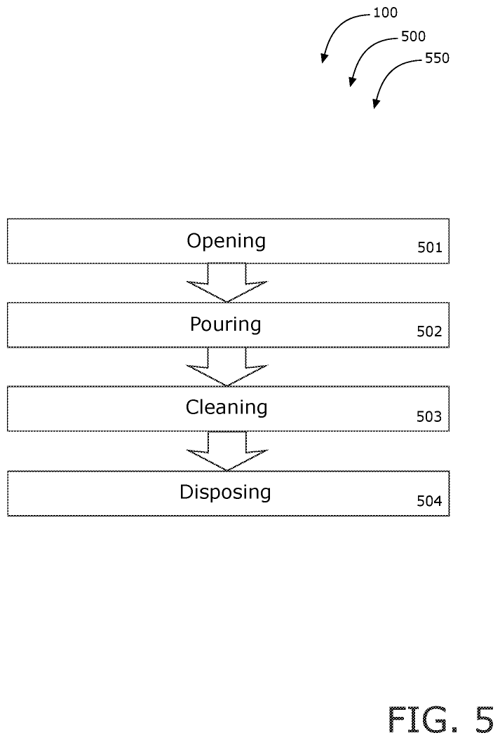

Referring now to FIG. 5 showing flowchart 550 illustrating method of use for single-serve oral rinse system 100 according to an embodiment of the present invention of FIGS. 1-4. As shown, method of use 500 may comprise the steps of step one 501, opening a packet of a flavored bleach composition; step two 502, pouring flavored bleach composition 110 into an oral irrigation device 180; step three 503, cleaning at least one oral surface; and step three 503, disposing of packaging (disposable packet 190).

Various inventive methods comprise providing any of the compositions described above. The composition is combined with an amount of water to make a water-flosser rinse. The user fills the water flosser with the fluid and then conducts a water flossing session. Typically, the rinse is non-toxic enough to be disposed of down a residential sink safely and legally.

Various embodiments of the invention use a mixture of an orally active germicide mixed with a flavoring such as mint, peppermint, wintergreen, anise, clove, eucalyptus, lemon, lime, honey, honey lemon, red fruit, grapefruit, orange, grape, cherry, cherry cola, berry, or cinnamon. Some mixtures include fluoride. In various embodiments of the invention, the germicide is any single or any combination of glutaraldehyde, chlorhexidine, diamines, alexidine, phenols, formaldehyde, glutaraldehyde, acridines, silver compounds, halogens, silver ions, hexachlorophene, benzalkonium chloride, or bleach.

The steps described in the method of use can be carried out in many different orders according to user preference. The use of “step of” should not be interpreted as “step for”, in the claims herein and is not intended to invoke the provisions of 35 U.S.C. § 112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary, and numerous modifications, variations, and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A method comprising providing a single-dose package comprising a mixture of an orally active germicide and a flavoring agent.

2. The method of claim 1, wherein the package contains an amount of mixture sufficient to form an effective concentration of germicide and flavoring agent for a single session of water flossing.

3. The method of claim 2, wherein the mixture comprises a powder, granule, gel, liquid, or tablet.

4. The method of claim 3, wherein the mixture further comprises a solid or liquid carrier.

5. The method of claim 4, wherein the mixture further comprises fluoride.

6. The method of claim 5 further comprising forming a water-flossing fluid by adding the mixture to 10 ounces of water.

7. The method of claim 6 further comprising placing the water-flossing fluid into a reservoir of a water-flossing device.

8. The method of claim 7, wherein the flavoring agent is any single one or any combination of anise, peppermint, clove, eucalyptus, lemon, lime, honey, honey lemon, red fruit, mint, grapefruit, orange, grape, cherry, cherry cola, berry, wintergreen, and cinnamon flavor.

9. The method of claim 8, wherein the germicide is any single one or any combination of glutaraldehyde, chlorhexidine, diamines, alexidine, phenols, formaldehyde, glutaraldehyde, acridines, silver compounds, halogens, silver ions, hexachlorophene, benzalkonium chloride, or bleach.

10. The method of claim 9, wherein the germicide is bleach.

11. The method of claim 10, wherein the germicide is sodium hypochlorite.

12. The method of claim 11, wherein the amount of mixture is one-half of a teaspoon.

13. The method of claim 5 further comprising forming a water-flossing fluid by adding the mixture to 10 ounces of water.

14. The method of claim 13 further comprising placing the water-flossing fluid into a reservoir of a water-flossing device.

15. The method of claim 14, wherein the flavoring agent is any single one or any combination of anise, peppermint, clove, eucalyptus, lemon, lime, honey, honey lemon, red fruit, mint, grapefruit, orange, grape, cherry, cherry cola, berry, wintergreen, and cinnamon flavor.

16. The method of claim 15, wherein the germicide is any single one or any combination of glutaraldehyde, chlorhexidine, diamines, alexidine, phenols, formaldehyde, glutaraldehyde, acridines, silver compounds, halogens, silver ions, hexachlorophene, benzalkonium chloride, or bleach.

17. The method of claim 16, wherein the germicide is bleach.

18. The method of claim 17, wherein the germicide is sodium hypochlorite.

19. The method of claim 18, wherein the amount of mixture is one-half of a teaspoon.
